(19) United States
(12) Patent Application Publication
(10) Pub. No.: US 2018/0120396 A1
(43) Pub. Date: May 3, 2018

(54) MAGNETIC RESONANCE IMAGING THERMOMETRY USING PROTON RESONANCE FREQUENCY AND T1 MEASUREMENTS

(71) Applicant: KONINKLIJKE PHILIPS N.V., EINDHOVEN (NL)

(72) Inventor: STEFFEN WEISS, EINDHOVEN (NL)

(21) Appl. No.: 15/561,095

(22) PCT Filed: Mar. 28, 2016
(86) PCT No.: PCT/EP2016/056742

§ 371 Date: Sep. 25, 2017

(30) Foreign Application Priority Data
Mar. 26, 2015 (EP) 15160990.6

(51) Int. Cl. G01R 33/48; G01R 33/50; G01R 33/56; A61B 5/01; A61B 5/055; A61B 18/02; A61B 18/12; A61B 18/18; A61B 18/20; A61B 34/00; A61B 90/00

(52) U.S. Cl. CPC G01R 33/4804; G01R 33/50; G01R 33/5608; A61B 5/015; A61B 5/055; A61B 2018/00714; A61B 18/12; A61B 18/1815; A61B 18/20; A61B 34/25; A61B 90/37; A61B 18/02

(57) ABSTRACT

The invention provides for a method of operating a medical instrument (100, 400, 500, 600), with magnetic resonance imaging system (102). The method comprises acquiring (202) equilibrium magnetization magnetic resonance imaging data (148) by controlling the magnetic resonance imaging system according to a T1 measuring magnetic resonance imaging protocol and calculating an equilibrium magnetization baseline image (156). The method further comprises repeatedly acquiring (206) the dynamic PRFS magnetic resonance data according to a proton resonance frequency shift magnetic resonance imaging protocol. The method further comprises repeatedly acquiring (208) magnetic resonance data portions (152) according to the T1 measuring magnetic resonance imaging protocol with a saturation preparation (804) at the start of the acquisition. The acquisition of the dynamic PRFS magnetic resonance data and the magnetic resonance data portions are interleaved. The method further comprises repeatedly reassembling (212) the resonance data portions into dynamic T1 magnetic resonance data. The method further comprises repeatedly calculating (214) a T1 map (158) using the reassembled dynamic T1 magnetic resonance data and the equilibrium magnetization image. The method further comprises repeatedly calculating (216) a PRFS phase calibration (160) using the dynamic PRFS magnetic resonance data and the T1 map. The method further comprises repeatedly calculating (218) a PRFS temperature map (162) using the dynamic PRFS magnetic resonance data and the PRFS phase calibration if the PRFS phase calibration has been calculated.

### TECHNICAL FIELD OF THE INVENTION

The invention relates to magnetic resonance imaging, in particular to magnetic resonance imaging thermometry.

### BACKGROUND OF THE INVENTION

Magnetic resonance thermometry may be used to determine either the absolute temperature of a volume or a change in temperature, depending upon the technique used. For determining the absolute temperature several magnetic resonance peaks are typically measured. Methods which measure changes in temperature are typically faster and have been used to take temperature measurements for guiding thermal treatments. For example Proton resonance frequency shift (PRFS or PRF) based MR thermometry may be employed to provide temperature maps rapidly and accurately. However, PRFS based methods rely on making an accurate phase calibration, which is in turn very susceptible to changes in the B0 field of the magnet.

The journal article Todd, N., Diakite, M., Payne, A. and Parker, D. L. (2013), Hybrid proton resonance frequency/T1 technique for simultaneous temperature monitoring in adipose and aqueous tissues. Magn Reson Med, 69: 62-70. doi: 10.1002/mrm.24228 describes a combined T1 and PRFS pulse sequence that a standard RF spoiled gradient echo sequence execute in a dynamic mode with two flip angels alternating every time frame.

### SUMMARY OF THE INVENTION

The invention provides for a medical instrument, a method of operating the medical instrument, and a computer program product in the independent claims. Embodiments are given in the dependent claims.

As will be appreciated by one skilled in the art, aspects of the present invention may be embodied as an apparatus, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer executable code embodied thereon.

Any combination of one or more computer readable medium(s) may be utilized. The computer readable medium may be a computer readable signal medium or a computer readable storage medium. A ‘computer-readable storage medium’ as used herein encompasses any tangible storage medium which may store instructions which are executable by a processor of a computing device. The computer-readable storage medium may be referred to as a computer-readable non-transitory storage medium. The computer-readable storage medium may also be referred to as a tangible computer readable medium. In some embodiments, a computer-readable storage medium may also be able to store data which is able to be accessed by the processor of the computing device. Examples of computer-readable storage media include, but are not limited to: a floppy disk, a magnetic hard disk drive, a solid state hard disk, flash memory, a USB thumb drive, Random Access Memory (RAM), Read Only Memory (ROM), an optical disk, a magneto-optical disk, and the register file of the processor. Examples of optical disks include Compact Disks (CD), Digital Versatile Disks (DVD), and Blu-Ray Disc (BD), for example CD-ROM, CD-RW, CD-R, DVD-ROM, DVD-RW, DVD-R, BD-R, or BD-RE disks. The term computer readable-storage medium also refers to various types of recording media capable of being accessed by the computer device via a network or communication link. For example a data may be retrieved over a modem, over the internet, or over a local area network. Computer executable code embodied on a computer readable medium may be transmitted using any appropriate medium, including but not limited to wireless, wireline, optical fiber cable, RF, etc., or any suitable combination of the foregoing.

A computer readable signal medium may include a propagated data signal with computer executable code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

‘Computer memory’ or ‘memory’ is an example of a computer-readable storage medium. Computer memory is any memory which is directly accessible to a processor. ‘Computer storage’ or ‘storage’ is a further example of a computer-readable storage medium. Computer storage is any non-volatile computer-readable storage medium. In some embodiments computer storage may also be computer memory or vice versa.

A ‘processor’ as used herein encompasses an electronic component which is able to execute a program or machine executable instruction or computer executable code. References to the computing device comprising “a processor” should be interpreted as possibly containing more than one processor or processing core. The processor may for instance be a multi-core processor. A processor may also refer to a collection of processors within a single computer system or distributed amongst multiple computer systems. The term computing device should also be interpreted to possibly refer to a collection or network of computing devices each comprising a processor or processors. The computer executable code may be executed by multiple processors that may be within the same computing device or which may even be distributed across multiple computing devices.

Computer executable code may comprise machine executable instructions or a program which causes a processor to perform an aspect of the present invention. Computer executable code for carrying out operations for aspects of the present invention may be written in any combination of one or more programming languages, including an object oriented programming language such as Java, Smalltalk, C++ or the like and conventional procedural programming languages, such as the “C” programming language or similar programming languages and compiled into machine executable instructions. In some instances the computer executable code may be in the form of a high level language or in a pre-compiled form and be used in conjunction with an interpreter which generates the machine executable instructions on the fly.

The computer executable code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described with reference to flowchart illustrations and/or block diagrams of methods, apparatus (systems) and computer program products according to embodiments of the invention. It will be understood that each block or a portion of the blocks of the flowchart, illustrations, and/or block diagrams, can be implemented by computer program instructions in form of computer executable code when applicable. It is further under stood that, when not mutually exclusive, blocks in different flowcharts, illustrations, and/or block diagrams may be combined. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

A ‘user interface’ as used herein is an interface which allows a user or operator to interact with a computer or computer system. A ‘user interface’ may also be referred to as a ‘human interface device.’ A user interface may provide information or data to the operator and/or receive information or data from the operator. A user interface may enable input from an operator to be received by the computer and may provide output to the user from the computer. In other words, the user interface may allow an operator to control or manipulate a computer and the interface may allow the computer indicate the effects of the operator's control or manipulation. The display of data or information on a display or a graphical user interface is an example of providing information to an operator. The receiving of data through a keyboard, mouse, trackball, touchpad, pointing stick, graphics tablet, joystick, gamepad, webcam, headset, gear sticks, steering wheel, pedals, wired glove, dance pad, remote control, and accelerometer are all examples of user interface components which enable the receiving of information or data from an operator.

A ‘hardware interface’ as used herein encompasses an interface which enables the processor of a computer system to interact with and/or control an external computing device and/or apparatus. A hardware interface may allow a processor to send control signals or instructions to an external computing device and/or apparatus. A hardware interface may also enable a processor to exchange data with an external computing device and/or apparatus. Examples of a hardware interface include, but are not limited to: a universal serial bus, IEEE 1394 port, parallel port, IEEE 1284 port, serial port, RS-232 port, IEEE-488 port, Bluetooth connection, Wireless local area network connection, TCP/IP connection, Ethernet connection, control voltage interface, MIDI interface, analog input interface, and digital input interface.

A ‘display’ or ‘display device’ as used herein encompasses an output device or a user interface adapted for displaying images or data. A display may output visual, audio, and or tactile data. Examples of a display include, but are not limited to: a computer monitor, a television screen, a touch screen, tactile electronic display, Braille screen,

Cathode ray tube (CRT), Storage tube, Bistable display, Electronic paper, Vector display, Flat panel display, Vacuum fluorescent display (VF), Light-emitting diode (LED) displays, Electroluminescent display (ELD), Plasma display panels (PDP), Liquid crystal display (LCD), Organic light-emitting diode displays (OLED), a projector, and Head-mounted display.

Medical image data is defined herein as two or three dimensional data that has been acquired using a medical imaging scanner. A medical imaging scanner is defined herein as an apparatus adapted for acquiring information about the physical structure of a patient and construct sets of two dimensional or three dimensional medical image data. Medical image data can be used to construct visualizations which are useful for diagnosis by a physician. This visualization can be performed using a computer.

Magnetic Resonance (MR) data is defined herein as being the recorded measurements of radio frequency signals emitted by atomic spins by the antenna of a Magnetic resonance apparatus during a magnetic resonance imaging scan. Magnetic resonance data is an example of medical image data. A Magnetic Resonance Imaging (MRI) image is defined herein as being the reconstructed two or three dimensional visualization of anatomic data contained within the magnetic resonance imaging data. This visualization can be performed using a computer.

Magnetic resonance data may comprise the measurements of radio frequency signals emitted by atomic spins by the antenna of a Magnetic resonance apparatus during a magnetic resonance imaging scan which contains information which may be used for magnetic resonance thermometry. Magnetic resonance thermometry functions by measuring changes in temperature sensitive parameters. Examples of parameters that may be measured during magnetic resonance thermometry are: the proton resonance frequency shift, the diffusion coefficient, or changes in the T1 and/or T2 relaxation time may be used to measure the temperature using magnetic resonance. The proton resonance frequency shift is temperature dependent, because the magnetic field that individual protons, hydrogen atoms, experience depends upon the surrounding molecular structure. An increase in temperature decreases molecular screening due to the temperature affecting the hydrogen bonds. This leads to a temperature dependence of the proton resonance frequency.

The proton density depends linearly on the equilibrium magnetization. It is therefore possible to determine temperature changes using proton density weighted images.

The relaxation times T1, T2, and T2-star (sometimes written as T2*) are also temperature dependent. The reconstruction of T1, T2, and T2-star weighted images can therefore be used to construct thermal or temperature maps.

The temperature also affects the Brownian motion of molecules in an aqueous solution. Therefore pulse sequences which are able to measure diffusion coefficients such as a pulsed diffusion gradient spin echo may be used to measure temperature.

One of the most useful methods of measuring temperature using magnetic resonance is by measuring the proton resonance frequency (PRF) shift of water protons. The resonance frequency of the protons is temperature dependent. As the temperature changes in a voxel the frequency shift will cause the measured phase of the water protons to change. The temperature change between two phase images can therefore be determined. This method of determining temperature has the advantage that it is relatively fast in comparison to the other methods.

An ‘ultrasound window’ as used herein encompasses a window which is effectively transparent to ultrasonic waves or energy. Typically a thin film or membrane is used as an ultrasound window. The ultrasound window may for example be made of a thin membrane of BoPET (Biaxially-oriented polyethylene terephthalate).

In one aspect the invention provides for a medical instrument. The medical instrument comprises a magnetic resonance imaging system for acquiring magnetic resonance data within an imaging zone. The medical instrument further comprises a memory storing machine-executable instructions. The memory further stores first pulse sequence commands, second pulse sequence commands, and third pulse sequence commands. Pulse sequence commands as used herein are either commands, which may be used for directly controlling a magnetic resonance imaging system or data, which may be converted into such commands. For example pulse sequences are typically defined in terms of timing diagrams. The data used to define a timing diagram may be converted into commands for controlling the magnetic resonance imaging system. The pulse sequence commands may also control data for controlling the operation of other instruments that may be used in conjunction with the magnetic resonance imaging system. For example the pulse sequence commands may also contain commands for controlling a temperature control system.

The first pulse sequence commands cause the magnetic resonance imaging system to acquire equilibrium magnetization magnetic resonance data according to a T1 measuring magnetic resonance imaging protocol. The term equilibrium magnetization magnetic resonance data is a label which refers to specific magnetic resonance data that is acquired according to the T1 measuring magnetic resonance imaging protocol. The second pulse sequence commands cause the magnetic resonance imaging system to acquire dynamic PRFS magnetic resonance data according to a proton residency frequency shift magnetic resonance imaging protocol. The abbreviation PRFS is used as an abbreviation for proton resonance frequency shift. The third pulse sequence commands cause the magnetic resonance imaging system to acquire dynamic T1 magnetic resonance data according to the T1 measuring magnetic resonance imaging protocol. The third pulse sequence commands further cause the magnetic resonance imaging system to acquire the T1 magnetic resonance data sequentially as a set of magnetic resonance data portions. The first pulse sequence commands causes the entire k-space of the T1 magnetic resonance imaging protocol to be acquired at once. The third pulse sequence commands cause the k-space data to be acquired in magnetic resonance data portions. For example a trajectory in k-space could be traced out for each of the magnetic resonance data portions that are acquired.

The medical instrument further comprises a processor for controlling the medical instrument. The processor for example may also be considered to be a controller. Execution of the machine-executable instructions causes the processor to acquire equilibrium magnetization magnetic resonance imaging data by controlling the magnetic resonance imaging system using the first pulse sequence commands. The T1 measuring magnetic resonance imaging protocol is a magnetic resonance imaging protocol that is used to measure the T1 value for each voxel that is imaged. Execution of the machine-executable instructions further cause the processor to calculate an equilibrium magnetization baseline image from the equilibrium magnetization magnetic resonance imaging data. For each voxel that is imaged the equilibrium magnetization is calculated and this may be represented as a image or as set of values in a two- or three-dimensional array of data.

Execution of the machine-executable instructions further cause the processor to repeatedly acquire the PRFS magnetic resonance data by controlling the magnetic resonance imaging system with the second pulse sequence commands. Execution of the machine-executable instructions further cause the processor to repeatedly acquire a magnetic resonance data portion by controlling the magnetic resonance imaging system with the third pulse sequence commands. The acquisition of the PRFS magnetic resonance data and the magnetic resonance data portion are interleaved. In other words the PRFS magnetic resonance data is acquired alternately with acquiring one portion of the set of magnetic resonance data portions. The magnetic resonance data portion belongs to the set of magnetic resonance data portions.

Execution of the machine-executable instructions further cause the processor to repeatedly reassemble the set of magnetic resonance data portions into the dynamic T1 magnetic resonance data after the complete set of magnetic resonance data portions is acquired. As the PRFS magnetic resonance data and the magnetic resonance data portion are alternately acquired eventually the complete set of magnetic resonance data portions will be acquired. At this point they are reassembled into the full dynamic T1 magnetic resonance data. Execution of the machine-executable instructions further cause the processor to calculate a T1 map using the reassembled dynamic T1 magnetic resonance data and the equilibrium magnetization image. The calculation of the T1 map is enabled by using the equilibrium magnetization image. The equilibrium magnetization image was reconstructed from data that was acquired before the first PRFS magnetic resonance data was acquired.

The acquisition of the PRFS magnetic resonance data therefore did not interfere with the measurement of the equilibrium magnetization baseline image. Execution of the machine-executable instructions further cause the processor to repeatedly calculate a PRFS phase calibration using the PRFS magnetic resonance data and the T1 map. The proton resonance frequency shift method of measuring temperature is very quick and accurate; however it is susceptible to B0 drift. The B0 drift is however something which occurs on a timescale that is long in comparison to the acquisition of magnetic resonance data. By repeatedly acquiring the T1 magnetic resonance data during the acquisition of normal PRFS magnetic resonance data the reassembled magnetic resonance data portions can be used to periodically recalibrate the PRFS method. Execution of the machine-executable instructions further cause the processor to calculate a PRFS temperature map using the PRFS magnetic resonance data and the PRFS phase calibration if the PRFS phase calibration has been calculated. In other examples the first PRFS phase calibration is calculated by using the first PRFS magnetic resonance data. For example a temperature distribution within the body could be assumed and used to calculate an initial calibration. For example, first dynamic PRFS magnetic resonance data can be acquired before actual heating of the tissue is performed can be used for initial PRFS phase calibration. In other examples the T1 data and the baseline magnetization from the equilibrium magnetization baseline image could be used to calculate a temperature from the T1 values that is then used to calculate a PRFS phase calibration initially.

This embodiment may be beneficial because it provides a stable way of measuring the temperature of a subject using the PRFS method of measuring temperature for magnetic resonance imaging.

In another embodiment execution of the machine-executable instructions may further cause the processor to display the PRFS temperature map on a display, to store it in a computer storage device, or to transmit it to another computer system via a network or other data transfer system.

In another embodiment execution of the instructions further cause the processor to repeatedly calculate a dynamic image from a chosen magnetic resonance data selected from the set of magnetic resonance data portions. The chosen magnetic resonance data is selected such that the chosen magnetic resonance data portion maximizes the longitudinal magnetization. Each time a magnetic resonance data portion is acquired measurements at different delays from the saturation preparation. The data acquired further in time from the saturation preparation has had more time for the longitudinal magnetization to recover. After the entire set of magnetic resonance data portions has been acquired and combined. The chosen magnetic resonance data can be removed from each of the magnetic resonance data portions. Those data is selected such that only those portions which maximize the longitudinal magnetization are selected (i.e. the data acquired with the largest delay from the saturation preparation). The selection of this data results in a dynamic image which has a longitudinal magnetization which is close to the equilibrium value. This in turn enables a direct comparison between the dynamic image and the equilibrium magnetization baseline image.

Execution of the machine-executable instructions further cause the processor to repeatedly detect a subject motion above a predetermined threshold using the equilibrium magnetization baseline image and the dynamic image. The magnetic resonance data portion is only a portion of the complete k-space; however a full set of magnetic resonance data portions may be used to reconstruct such an image, which may be compared to the equilibrium magnetization baseline image.

In another embodiment the dynamic image is calculated from the data acquired late in the T1 relaxation curve, i.e., from data at the ends of the data portions. Late in the T1 relaxation cure herein means that the magnetic resonance data portion that is selected to reconstruct the dynamic image is the last one acquired or one of the last several that is acquired. The longitudinal magnetization has had the chance to almost recover which enables the dynamic image to be compared directly to the equilibrium magnetization baseline image. The equilibrium magnetization image may also be referred to as the M₀ image herein.

Calculating the dynamic image from data acquire late in the T1 relaxation curve may be advantageous because the resulting contrast of the dynamic image will be similar to that of the equilibrium image. A variety of different techniques which are known in the art may be used to detect if the subject has moved above a predetermined threshold beyond when the equilibrium magnetization baseline image was acquired.

As the magnetic resonance data portion is acquired relatively often this enables rapid detection of the motion of a subject. This may enable rapid determination if the PRFS temperature map is no longer valid. Execution of the machine-executable instructions further cause the processor to repeatedly reacquire the equilibrium magnetization magnetic resonance data by controlling the magnetic resonance imaging system using the first pulse sequence commands if the subject motion is detected. In some instances this may require pausing the acquisition of magnetic resonance data altogether so as to enable the equilibrium magnetization to recover to its equilibrium state. Execution of the machine-executable instructions further cause the processor to repeatedly recalculate the equilibrium magnetization baseline image from the equilibrium magnetization magnetic resonance imaging data if the subject motion is detected. In some instances this step may also involve calculating a new PRFS phase calibration. This embodiment may have the benefit that the motion of a subject can be quickly detected and corrections to the PRFS temperature map can be made. This may lead to more accurate PRFS temperature maps.

In another embodiment the subject motion is detected using a cross correlation algorithm.

In another embodiment the subject motion is detected using a rigid body motion detection algorithm.

In another embodiment the subject motion is detected using an elastic registration algorithm.

In another embodiment the subject motion is detected using an optical flow algorithm.

In another embodiment the medical instrument further comprises a temperature control system for modifying the temperature within a target zone. The target zone is within the imaging zone.

In another embodiment the temperature control system is a high-intensity focused ultrasound system.

In another embodiment the temperature control system is a radio-frequency tissue heating system.

In another embodiment the temperature control system is a microwave applicator.

In another embodiment the temperature control system is a cryo-ablator.

In another embodiment the temperature control system is a laser.

In another embodiment execution of the machine-executable instructions further cause the processor to receive temperature control system commands that cause the temperature control system to modify the temperature of the target zone. The temperature control system commands may be commands that the processor uses to directly control the temperature control system or it may be commands or data which is used to generate commands which are used to control the temperature control system. Execution of the machine-executable instructions further causes the processor to repeatedly modify the temperature control system commands using the PRFS temperature map. These steps effectively form a control loop for controlling the temperature control system. For example the temperature control system commands may specify that regions or particular locations within the subject are heated to a particular temperature for a duration of time. The PRFS temperature map can be used as feedback to more accurately control the temperature control system to follow the temperature control system commands.

In another embodiment the medical instrument further comprises a user interface with a display. Execution of the machine-executable instructions further cause the processor to display the PRFS temperature map on the display. Execution of the machine-executable instructions further causes the processor to receive control data from the user interface. The control data may for example comprise commands to heat or not heat particular regions of the subject. The user control data may also include data which modifies the behavior of the temperature control system. Execution of the machine-executable instructions further cause the processor to modify the temperature control system commands using the user control data.

In another embodiment execution of the machine-executable instructions further causes the processor to control the temperature control system with the temperature control system commands.

In another embodiment the T1 magnetic resonance imaging protocol is a saturation recovery look-locker magnetic resonance imaging protocol.

In another embodiment execution of the magnetic resonance imaging system causes the processor to perform after a predetermined time interval: reacquire the equilibrium magnetization magnetic resonance imaging data by controlling the magnetic resonance imaging system using the first pulse sequence commands, and recalculating an equilibrium magnetization image from the equilibrium magnetization magnetic resonance imaging data if the subject motion is detected. In some examples the PRFS phase calibration may also be recalculated. In this example the equilibrium magnetization image is acquired and calculated after a passage of time. Even though the subject motion has for example not been detected, it may be beneficial to nonetheless periodically check to ensure that the equilibrium magnetization image is still accurate.

In another embodiment the third pulse sequence commands cause the magnetic resonance imaging system to perform a saturation preparation at the start of the acquisition of each magnetic resonance data portion. A saturation preparation as used here encompasses a radio frequency pulse and gradient pulses which reduces the longitudinal magnetization to zero and spoils all transverse magnetization. In the literature the saturation preparation is sometimes referred to as a “saturation preparation pulse.”

This may be beneficial because the saturation radio-frequency preparation reduces the longitudinal magnetization to zero, which effectively negates the effect of performing a PRFS measurement immediately before making the T1 measurement.

In another aspect the invention provides for a method of operating the medical instrument. The medical instrument comprises a magnetic resonance imaging system for acquiring magnetic resonance data within an imaging zone. The method comprises the step of acquiring equilibrium magnetization magnetic resonance imaging data by controlling the magnetic resonance imaging system using first pulse sequence commands. The first pulse sequence commands cause the magnetic resonance imaging system to acquire equilibrium magnetization magnetic resonance data according to a T1 measuring magnetic resonance imaging protocol. The method further comprises calculating an equilibrium magnetization baseline image from the equilibrium magnetization magnetic resonance imaging data.

The method further comprises repeatedly acquiring the PRFS magnetic resonance data by controlling the magnetic resonance imaging system with second pulse sequence commands. The second pulse sequence commands cause the magnetic resonance imaging system to acquire dynamic PRFS magnetic resonance data according to a proton resonance frequency shift magnetic resonance imaging protocol. The method further comprises repeatedly acquiring a magnetic resonance data portion by controlling the magnetic resonance imaging system with third pulse sequence commands. The third pulse sequence commands cause the magnetic resonance imaging system to acquire dynamic T1 magnetic resonance data according to a T1 measuring magnetic resonance imaging protocol.

The third pulse sequence commands further cause the magnetic resonance imaging system to acquire the dynamic T1 magnetic resonance data sequentially as a set of magnetic resonance data portions. The acquisition of the dynamic PRFS magnetic resonance data and the magnetic resonance data portion are interleaved. The magnetic resonance data portion belongs to the set of magnetic resonance data portions. The method further comprises repeatedly reassembling the set of magnetic resonance data portions into the dynamic T1 magnetic resonance data after the complete set of magnetic resonance data portions is acquired. The method further comprises repeatedly calculating a T1 map using the reassembled dynamic T1 magnetic resonance data and the equilibrium magnetization baseline image.

The method further comprises repeatedly calculating a PRFS phase calibration using the dynamic PRFS magnetic resonance data and the T1 map. The method further comprises calculating a PRFS temperature map using the PRFS magnetic resonance data and the PRFS phase calibration if the PRFS phase calibration has been calculated.

The method further comprises repeatedly calculating a dynamic image from the magnetic resonance data portion. This is done after each magnetic resonance data portion is acquired. The method further comprises repeatedly detecting a subject motion above a predetermined threshold using equilibrium magnetization baseline image and the dynamic image. The method further comprises repeatedly reacquiring the equilibrium magnetization magnetic resonance imaging data by controlling the magnetic resonance imaging system using the first pulse sequence commands if the subject motion is detected. The method further comprises repeatedly recalculating the equilibrium magnetization baseline image from the equilibrium magnetization magnetic resonance imaging data if the subject motion is detected.

In another embodiment the method further comprises a correction for motion which may occur between the M0 scan (a zero magnetization scan) and the dynamic T1 acquisitions by comparison of each latest image acquired during the T1 relaxation after saturation to the equilibrium magnetization baseline image, and by reacquisition of the equilibrium magnetization data after pausing the dynamic acquisition in case of motion exceeding a user-defined limit

In another aspect the invention provides for a computer program product comprising machine-executable instructions for execution by a processor controlling a medical instrument. The medical instrument comprises a magnetic resonance imaging system for acquiring magnetic resonance data within an imaging zone. Execution of the machine-executable instructions causes the processor to acquire equilibrium magnetization magnetic resonance imaging data by controlling the magnetic resonance imaging system using the first pulse sequence commands. The first pulse sequence commands cause the magnetic resonance imaging system to acquire the equilibrium magnetization magnetic resonance data according to a T1 measuring magnetic resonance imaging protocol.

Execution of the machine-executable instructions further cause the processor to calculate an equilibrium magnetization baseline image from the equilibrium magnetization magnetic resonance imaging data. Execution of the machine-executable instructions causes the processor to repeatedly acquire dynamic PRFS magnetic resonance data by controlling the magnetic resonance imaging system with the second pulse sequence commands. The second pulse sequence commands cause the magnetic resonance imaging system to acquire the dynamic PRFS magnetic resonance data according to a proton resonance frequency shift magnetic resonance imaging protocol. Execution of the machine-executable instructions further cause the processor to repeatedly acquire a magnetic resonance data portion by controlling the magnetic resonance imaging system with third pulse sequence commands. The third pulse sequence commands cause the magnetic resonance imaging system to acquire dynamic T1 magnetic resonance data according to the T1 measuring magnetic resonance imaging protocol. The third pulse sequence commands further cause the magnetic resonance imaging system to acquire the dynamic T1 magnetic resonance data sequentially as a set of magnetic resonance data portions. The acquisition of the dynamic PRFS magnetic resonance data and the magnetic resonance data portion are interleaved. The magnetic resonance data portion belongs to the set of magnetic resonance data portions.

Execution of the machine-executable instructions further cause the processor to repeatedly reassemble the set of magnetic resonance data portions into the dynamic T1 magnetic resonance data after the complete set of magnetic resonance data portions is acquired. Execution of the machine-executable instructions further cause the processor to repeatedly calculate a T1 map using the reassembled dynamic T1 magnetic resonance data and the equilibrium magnetization image. Execution of the machine-executable instructions further cause the processor to repeatedly calculate a PRFS phase calibration using the dynamic PRFS magnetic resonance data and the T1 map. Execution of the machine-executable instructions further cause the processor to calculate a PRFS temperature map using the dynamic PRFS magnetic resonance data and the PRFS phase calibration if the PRFS phase calibration has been calculated.

It is understood that one or more of the aforementioned embodiments of the invention may be combined as long as the combined embodiments are not mutually exclusive.

### BRIEF DESCRIPTION OF THE DRAWINGS

In the following preferred embodiments of the invention will be described, by way of example only, and with reference to the drawings in which:

FIG. 1 illustrates an example of a medical instrument;

FIG. 2 shows a flow chart which illustrates a method of operating the medical instrument of FIG. 1, FIG. 4, FIG. 5, or FIG. 6;

FIG. 3 shows a flow chart which illustrates a further method of operating the medical instrument of FIG. 1, FIG. 4, FIG. 5, or FIG. 6;

FIG. 4 illustrates a further example of a medical instrument;

FIG. 5 illustrates a further example of a medical instrument;

FIG. 6 illustrates a further example of a medical instrument;

FIG. 7. shows a flow chart which illustrates a further method of operating the medical instrument of FIG. 4, FIG. 5, or FIG. 6;

FIG. 8 illustrates a combined pulse sequence protocol; and

FIG. 9 illustrate the k-space sampling for the first pulse sequence commands and the second pulse sequence commands.

### DETAILED DESCRIPTION OF THE EMBODIMENTS

Like numbered elements in these figures are either equivalent elements or perform the same function. Elements which have been discussed previously will not necessarily be discussed in later figures if the function is equivalent.

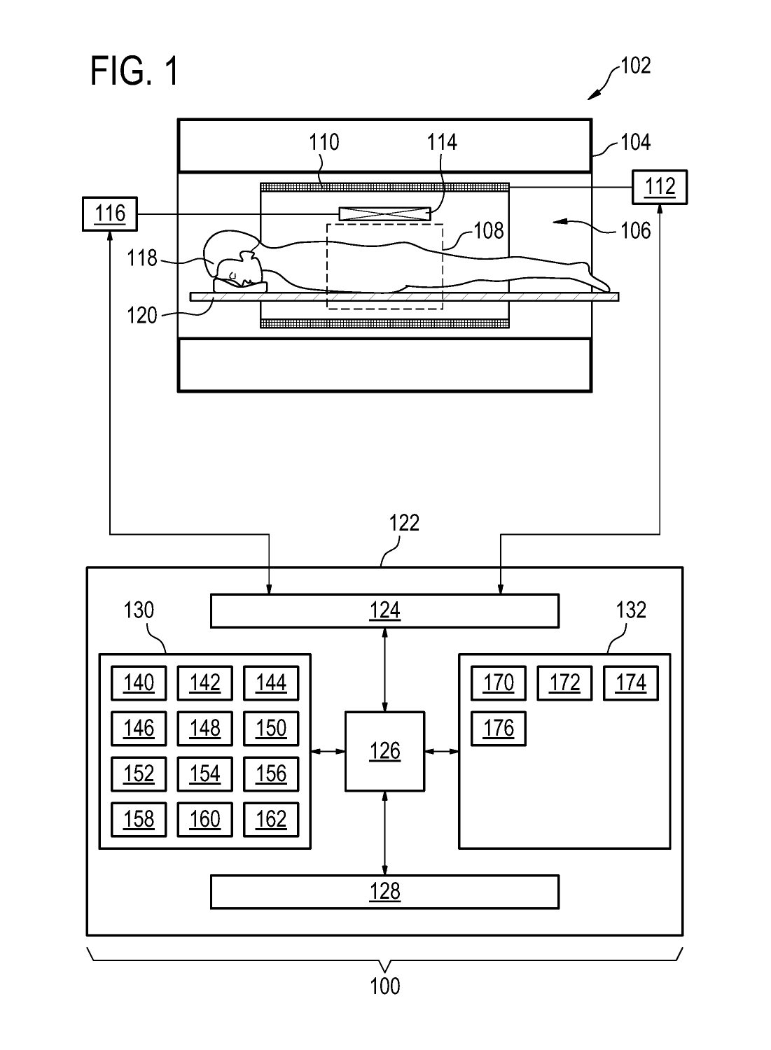

FIG. 1 illustrates an example of medical instrument. The medical instrument 100 comprises a magnetic resonance imaging system 102. The magnetic resonance imaging system 102 is shown as comprising a magnet 104. The magnet 104 is a cylindrical type superconducting magnet with a bore 106 through the center of it. The magnet 104 has a liquid helium cooled cryostat with superconducting coils. It is also possible to use permanent or resistive magnets. The use of different types of magnets is also possible for instance it is also possible to use both a split cylindrical magnet and a so called open magnet. A split cylindrical magnet is similar to a standard cylindrical magnet, except that the cryostat has been split into two sections to allow access to the iso-plane of the magnet, such magnets may for instance be used in conjunction with charged particle beam therapy. An open magnet has two magnet sections, one above the other with a space in-between that is large enough to receive a subject: the arrangement of the two sections area similar to that of a Helmholtz coil. Open magnets are popular, because the subject is less confined. Inside the cryostat of the cylindrical magnet there is a collection of superconducting coils. Within the bore of the cylindrical magnet there is an imaging zone 108 where the magnetic field is strong and uniform enough to perform magnetic resonance imaging.

Also within the bore of the magnet is a magnetic field gradient coil 110 which is used for acquisition of magnetic resonance data to spatially encode magnetic spins within an imaging zone of the magnet. The magnetic field gradient coil 110 is connected to a magnetic field gradient coil power supply 112. The magnetic field gradient coil is representative. Typically magnetic field gradient coils contain three separate sets of coils for spatially encoding in three orthogonal spatial directions. A magnetic field gradient power supply 112 supplies current to the magnetic field gradient coils. The current supplied to the magnetic field coils is controlled as a function of time and may be ramped and/or pulsed.

Adjacent the imaging zone 108 is a radio-frequency coil 114. The radio-frequency coil 114 is connected to a radio-frequency transceiver 116. Also within the bore of the magnet 106 is a subject 118 that is reposing on a subject support 120 and is partially within the imaging zone 108.

Adjacent to the imaging zone 108 is a radio-frequency coil 114 for manipulating the orientations of magnetic spins within the imaging zone 108 and for receiving radio transmissions from spins also within the imaging zone 108. The radio-frequency coil 114 may contain multiple coil elements. The radio-frequency coil 114 may also be referred to as a channel or an antenna. The radio-frequency coil is connected to a radio frequency transceiver 116. The radio-frequency coil 114 and radio frequency transceiver 116 may be replaced by separate transmit and receive coils and a separate transmitter and receiver. It is understood that the radio-frequency coil 114 and the radio-frequency transceiver 116 are representative. The radio-frequency coil 114 is intended to also represent a dedicated transmit antenna and a dedicated receive antenna. Likewise the transceiver 116 may also represent a separate transmitter and a separate receiver.

The magnetic field gradient coil power supply 112 and the radio-frequency transceiver 116 are connected to a hardware interface 124 of a computer system 122. The computer system 122 further comprises a processor 126. The processor 126 is connected to the hardware interface 124. The hardware interface 124 enables the processor 126 to send and receive data and commands to the magnetic resonance imaging system 102. The computer system 122 further comprises a user interface 128, computer storage 130 and computer memory 132.

The computer storage 130 is shown as containing first 140, second 142, and third 144 pulse sequence commands. The first pulse sequence commands 140 cause the magnetic resonance imaging system to acquire equilibrium magnetization magnetic resonance data according to a T1 measuring magnetic resonance imaging protocol. The second pulse sequence commands 142 cause the magnetic resonance imaging system to acquire dynamic PRFS magnetic resonance data according to a proton resonance frequency shift magnetic resonance imaging protocol. The third pulse sequence commands cause the magnetic resonance imaging system to acquire dynamic T1 magnetic resonance data 154 according to the T1 measuring magnetic resonance imaging protocol. The third pulse sequence commands 144 further causes the magnetic resonance imaging system 102 to acquire the dynamic T1 magnetic resonance data 154 sequentially as a set of magnetic resonance data portions 152. The computer storage 130 is further shown as containing equilibrium magnetization magnetic resonance data 148 that was acquired by controlling the magnetic resonance imaging system 102 with the first pulse sequence commands 140. The computer storage 130 is further shown as containing dynamic PRFS magnetic resonance data 150 that was acquired by controlling the magnetic resonance imaging system 102 with the second pulse sequence commands 142. The computer storage 130 is further shown as containing a magnetic resonance data portion 152 that was acquired by controlling the magnetic resonance imaging system 102 with the third pulse sequence commands 144. The computer storage 130 is further shown as containing the reassembled dynamic T1 magnetic resonance data 154 that was assembled from sequentially acquired magnetic resonance data portions 152. The computer storage 130 is further shown as containing an equilibrium magnetization baseline image 156 that was reconstructed from the equilibrium magnetization magnetic resonance data 148. The computer storage 130 is further shown as containing a T1 map 158 that was reconstructed from the equilibrium magnetization baseline image 156 and the reassembled dynamic T1 magnetic resonance data 154. The computer storage 130 is further shown as containing a PRFS phase calibration 160 that was calculated from the T1 map 158 and the dynamic PRFS magnetic resonance data 150. The computer storage 130 is further shown as containing a PRFS temperature map 162 that was calculated using the PRFS phase calibration 160 and the later acquired dynamic PRFS magnetic resonance data 150.

The computer memory 132 is shown as containing a control module 170. The control module 170 comprises computer-executable instructions which enable the processor 126 to control the operation and function of the medical instrument 100. The computer memory 132 is further shown as containing an image reconstruction module 172 that enables the processor 126 to process the various magnetic resonance data 148, 150, 152, 154, into various images or maps 156, 158, 160, 162. The computer memory 132 is further shown as containing an image processing module 174 which enables the processor 126 to manipulate and perform calculations or operations on the various images or maps. The computer memory 132 is further shown as containing a temperature mapping module 176. The temperature mapping module enables the processor 126 to apply T1 temperature mapping techniques and/or a PRFS temperature mapping technique. The contents of the computer storage 130 and the computer memory 132 may duplicate each other or the contents of either may be exchanged.

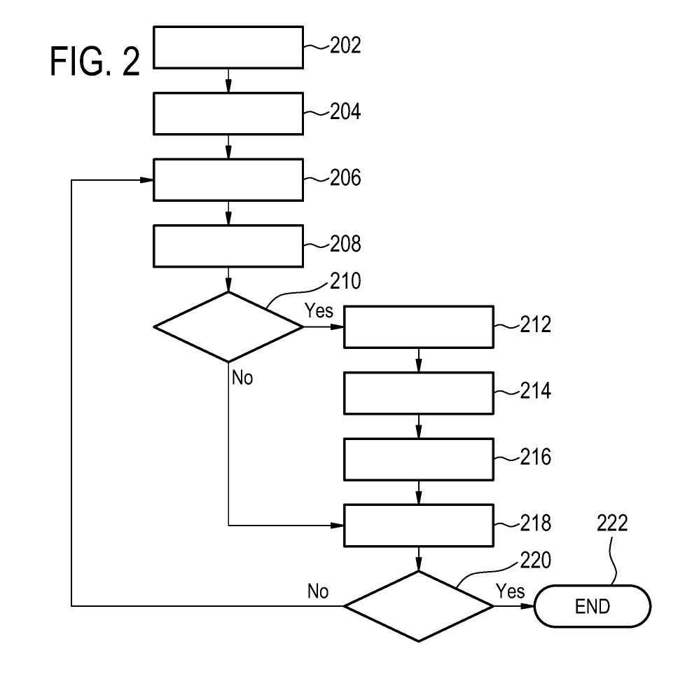

FIG. 2 shows a flowchart which illustrates an example of a method of operating the medical instrument 100 of FIG. 1. First in step 202 the processor 126 controls the magnetic resonance imaging system 102 to acquire the equilibrium magnetization magnetic resonance data 148 by controlling the magnetic resonance imaging system 102 with the first pulse sequence commands 140. Next in step 204 an equilibrium magnetization baseline image 156 is calculated from the equilibrium magnetization magnetic resonance imaging data 148. Next in step 206 the dynamic PRFS magnetic resonance data 150 is acquired by controlling the magnetic resonance imaging system 102 with the second pulse sequence commands 142. In step 208 a magnetic resonance data portion is acquired by controlling the magnetic resonance imaging system 102 with the third pulse sequence commands 144. The acquisition of the PRFS magnetic resonance data 150 and the magnetic resonance data portion 152 are interleaved. Step 210 is a decision box. The question for step 210 is the complete set of magnetic resonance data portions acquired. If no, then the method proceeds onto step 218 which is described below. If the answer is yes then the method proceeds onto step 212. In step 212 the set of magnetic resonance data portions is reassembled into the dynamic T1 magnetic resonance data 154.

Next in step 214 a T1 map is calculated using the reassembled dynamic T1 magnetic resonance data 154 and the equilibrium magnetization baseline image 156. Next in step 216 a PRFS phase calibration is calculated using the PRFS magnetic resonance data and the T1 map 154. Step 216 describes how the PRFS phase calibration is periodically replaced or re-calibrated using the T1 map and the equilibrium magnetization baseline image. There are a variety of different ways in which the initial PRFS phase calibration could be performed. In other examples the first time that the dynamic PRFS magnetic resonance data is acquired this is used for the calibration. The initial calibration of PRFS methods is well known and is therefore not discussed in detail here. A large number of variants in how to initially calculate the PRFS phase calibration could be performed by making slight modifications to the method described herein. The flowchart in FIG. 2 is intended to illustrate how the PRFS phase calibration could be periodically updated using the T1 map that is calculated.

In step 218 the PRFS temperature map is calculated. After step 218 the method proceeds to step 220 which is another decision box. In step 220 the question is the protocol complete. If the answer is yes then the method proceeds to step 222, which is the end of the protocol. If the answer to the question is no then the method proceeds back to step 206 where the acquisition of the interleaved PRFS magnetic resonance data and the magnetic resonance data portion begins again. The inclusion of a question block 220 is also illustrated. The method in FIG. 2 could be modified by breaking out from the flow at any time to end the procedure. The inclusion of steps 220 and 222 are intended only to be illustrative.

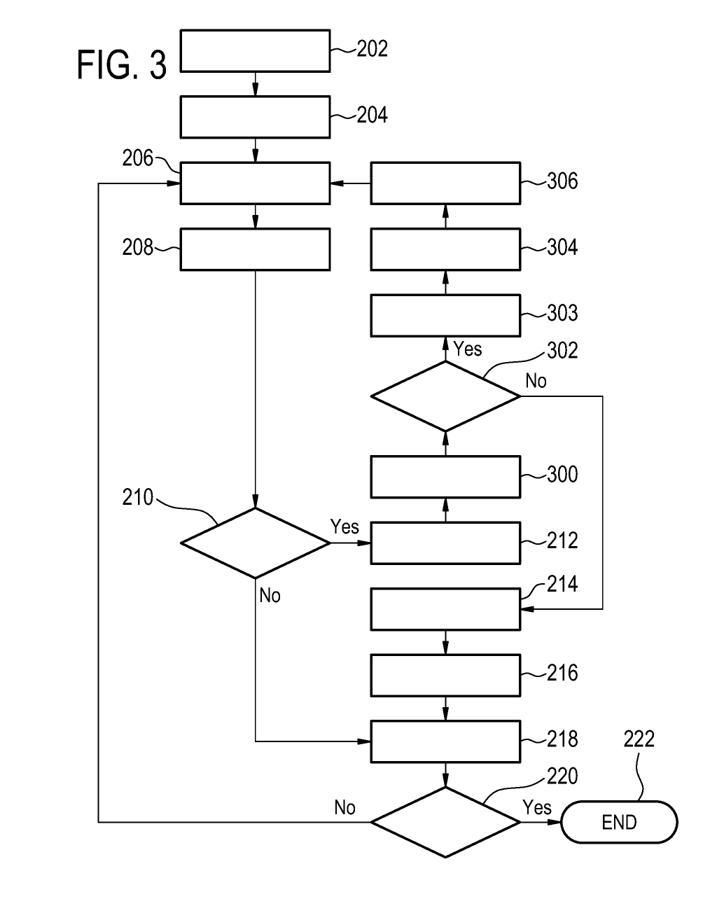

FIG. 3 shows a flowchart which illustrates a further example of a method of controlling the medical instrument of FIG. 1. The method shown in FIG. 3 is similar to that shown in FIG. 2. The method steps where the numbers are duplicated are equivalent steps. In this method the steps of the method shown in FIG. 2 have been modified. In this method the step 212 does not proceed directly onto step 214. After step 212 is performed the method proceeds onto step 300. In step 300 a dynamic image is calculated from a subset of all magnetic resonance data portions 152 characterized by being acquired late in the T1 relaxation curve. The magnetic resonance data portion is a trajectory in k-space that is a portion of what the k-space has been sampled for the equilibrium magnetization magnetic resonance data.

Next in step 302 a decision box is used to question if subject motion above a predetermined threshold is detected using the equilibrium magnetization baseline image 156 and the dynamic image. This for example may be calculated from the data acquired late in the T1 relaxation curve. If no motion is detected then the method goes from step 302 back to step 214; if motion is detected then the method proceeds onto step 303. Step 303 is an optional step between steps 302 and 304. The step 304 is a delay where the magnetic resonance imaging system is paused to allow the magnetization to recover to its equilibrium value. The pause may be for example be at least 3 to 5 time the T1 value.

In step 304 the equilibrium magnetization magnetic resonance data 148 is reacquired by controlling the magnetic resonance imaging system using the first pulse sequence commands 140. In some instances it may be beneficial to wait for the equilibrium magnetization within the subject to recover. This may entail a delay of a number of seconds, for example the method could be paused for a period of five seconds or so. Next in step 306 the equilibrium magnetization baseline image 156 is recalculated from the equilibrium magnetization magnetic resonance imaging data 148 that has just been reacquired.

After step 306 the method proceeds directly onto step 206 which is in fact to measure the PRFS magnetic resonance data. After performing step 206 the recalculation of the PRFS phase calculation is performed. There are a large number of variations as to how the PRFS phase calibration could in fact be recalculated. The recalculation of the PRFS phase calibration is therefore not detailed within FIG. 3.

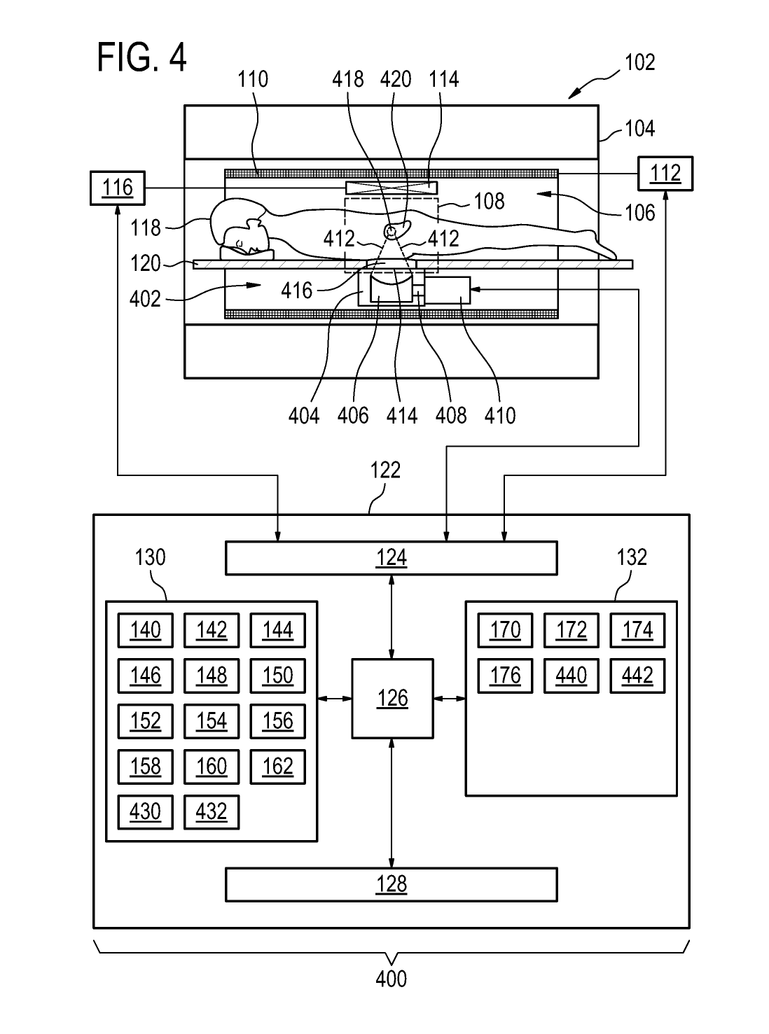

FIG. 4 shows a further example of a medical instrument 400. The medical instrument shown in FIG. 4 is similar to that shown in FIG. 1 except FIG. 4 also includes a high-intensity focused ultrasound system 402. The high-intensity focused ultrasound system 402 is an example of a temperature control system for modifying the temperature within a target zone 420.

FIG. 4 illustrates a further example of medical instrument 400. The example shown in FIG. 4 comprises a temperature treatment system which is a high-intensity focused ultrasound system 402. The high-intensity focused ultrasound system comprises a fluid-filled chamber 404. Within the fluid-filled chamber 404 is an ultrasound transducer 406. Although it is not shown in this FIG. the ultrasound transducer 406 may comprise multiple ultrasound transducer elements each capable of generating an individual beam of ultrasound. This may be used to steer the location of a sonication point 418 electronically by controlling the phase and/or amplitude of alternating electrical current supplied to each of the ultrasound transducer elements.

The ultrasound transducer 406 is connected to a mechanism 408 which allows the ultrasound transducer 406 to be repositioned mechanically. The mechanism 408 is connected to a mechanical actuator 410 which is adapted for actuating the mechanism 408. The mechanical actuator 410 also represents a power supply for supplying electrical power to the ultrasound transducer 406. In some examples the power supply may control the phase and/or amplitude of electrical power to individual ultrasound transducer elements. In some examples the mechanical actuator/power supply 410 is located outside of the bore 104 of the magnet 102.

The ultrasound transducer 406 generates ultrasound which is shown as following the path 412. The ultrasound 412 goes through the fluid-filled chamber 408 and through an ultrasound window 414. In this example the ultrasound then passes through a gel pad 416. The gel pad is not necessarily present in all examples but in this example there is a recess in the subject support 120 for receiving a gel pad 416. The gel pad 416 helps couple ultrasonic power between the transducer 406 and the subject 118. After passing through the gel pad 416 the ultrasound 412 passes through the subject 118 and is focused to a sonication point 418. The sonication point 418 is being focused within a target zone 420. The sonication point 418 may be moved through a combination of mechanically positioning the ultrasonic transducer 406 and electronically steering the position of the sonication point 418 to treat the entire target zone 420. Such a medical instrument 400 may be used to treat tissues which are at least partially fat. Examples include, but are not limited to: breast tissue, tissue in the pelvic cavity, and tissue in the abdominal cavity.

The high-intensity focused ultrasound system 402 is shown as being also connected to the hardware interference 124 of the computer system 122. The computer system 122 and the contents of its storage 130 and memory 132 are equivalent to that as shown in FIG. 1.

The computer storage 130 is shown as additionally containing a dynamic image 430 that was reconstructed from the magnetic resonance data portion 152. The computer storage 130 is further shown as containing temperature control system commands 432 that the processor 126 may use to control the high-intensity focused ultrasound system 402.

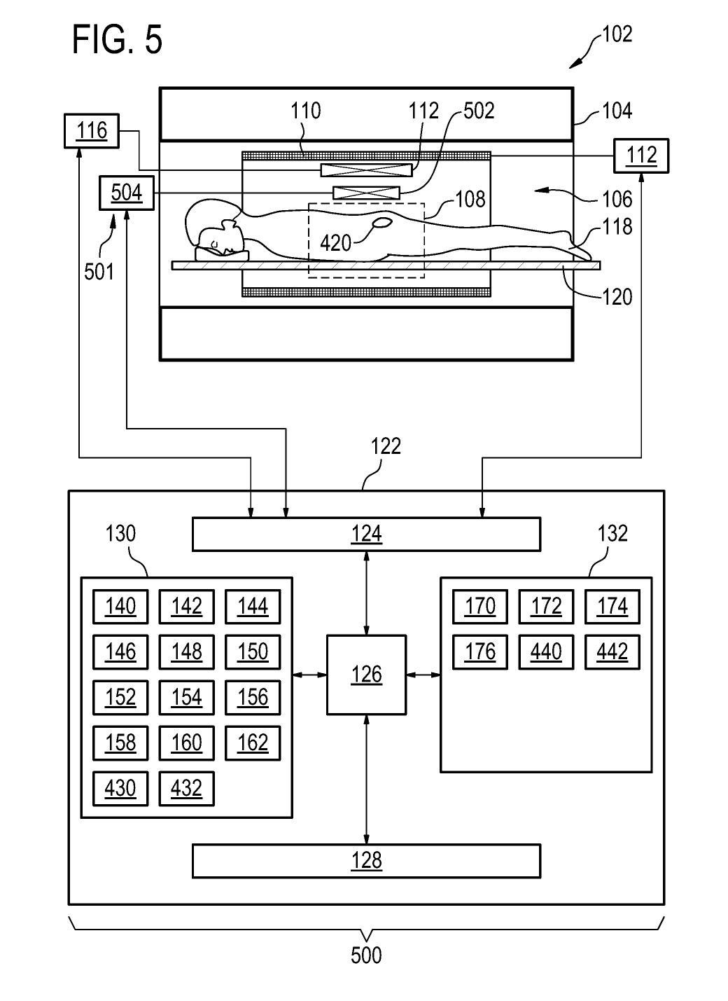

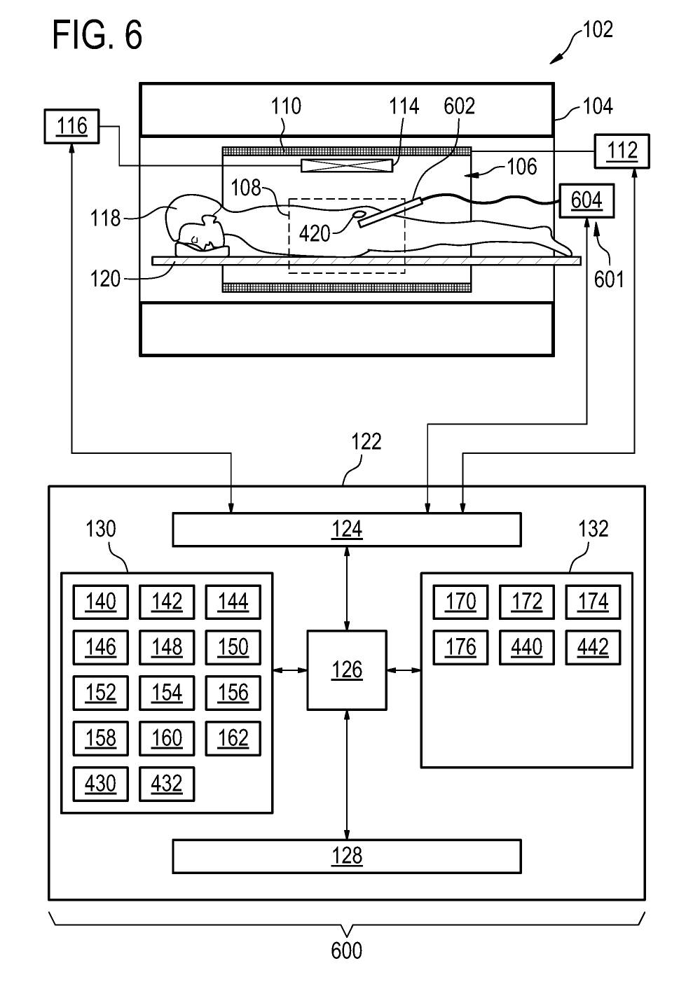

The computer memory 132 is further shown as containing a motion detection module 440 that is able to compare the dynamic image 430 to the equilibrium magnetization baseline image 156 in order to detect motion of the subject 118. The medical instrument 400 shown in FIG. 4 and also the subsequent medical instruments shown in FIGS. 5 and 6 are also able to perform the methods illustrated in FIGS. 2 and 3.

The computer memory 132 is further shown as containing a temperature control system command modification module 442 that is able to modify the temperature control system commands 432 using the PRFS temperature map 162. Using the PRFS temperature map 162 the temperature control system command modification module 442 forms a closed control loop for controlling the high-intensity focused ultrasound system 402. The software and control systems described for FIG. 4 are also generally applicable to other types of temperature control systems. It is understood that in the following FIGS. 5 and 6 modifications to the software may be made so that the software described in FIG. 4 is also applicable to FIGS. 5 and 6.

FIG. 5 shows a further example of medical instrument 500. The example shown in FIG. 5 is similar to that shown in FIG. 4. The computer system 122 of FIG. 5 is equivalent to the computer system 122 shown in FIGS. 3 and 4 also. The contents of the computer storage 130 and the computer memory 132 are also equivalent to the computer storage 130 and the computer memory 132 as shown in FIGS. 1, 3, and 4. In the example shown in FIG. 5 a radio-frequency tissue heating system 501 is used as the temperature treatment system. The radio-frequency temperature treatment system 501 comprises an antenna 502 and a radio-frequency transmitter 504. The antenna 502 is in the vicinity of target zone 420. Radio-frequency energy generated by the transmitter 504 and radiated by the antenna 502 is used to selectively heat the target zone 420. In this example the radio-frequency transmitter 504 is shown as being connected to the hardware interface 124. The processor 126 and the contents of the computer storage 130 and the computer memory 132 are used to control the radio-frequency transmitter 504 in a manner equivalent to the way the high-intensity focused ultrasound system 402 of FIG. 4 is controlled by the processor 124.

FIG. 6 shows a further example of a medical instrument. In this example a thermal treatment system 601 is shown. There is an applicator 602 which has been inserted into the subject 112. Near the tip of the applicator 602 is the treatment zone 420. The thermal treatment system 602 here is representative of a generic tissue heating system and may for example be either a microwave or RF applicator, a cryo-ablator, or a laser. The applicator 602 may be adapted for supplying microwave or RF energy for delivering heat, a cryogenic substance to the subject 112 or may be adapted for delivering light into the target zone 420 to produce heat. Likewise the supply system 604 may be a microwave or RF power supply, a supply system with a cryogenic or cooling fluid, or it may be a light power supply. The thermal treatment system 601 is shown as being connected to the hardware interface 124 of the computer system 132. The contents of the computer storage 130 and the computer memory 132 are equivalent to the examples shown in FIGS. 1, 3, 4, and 5. The instructions and computer code contained therein allow the processor 124 to control the thermal treatment system 601 in a manner equivalent to the examples shown in FIGS. 4 and 5.

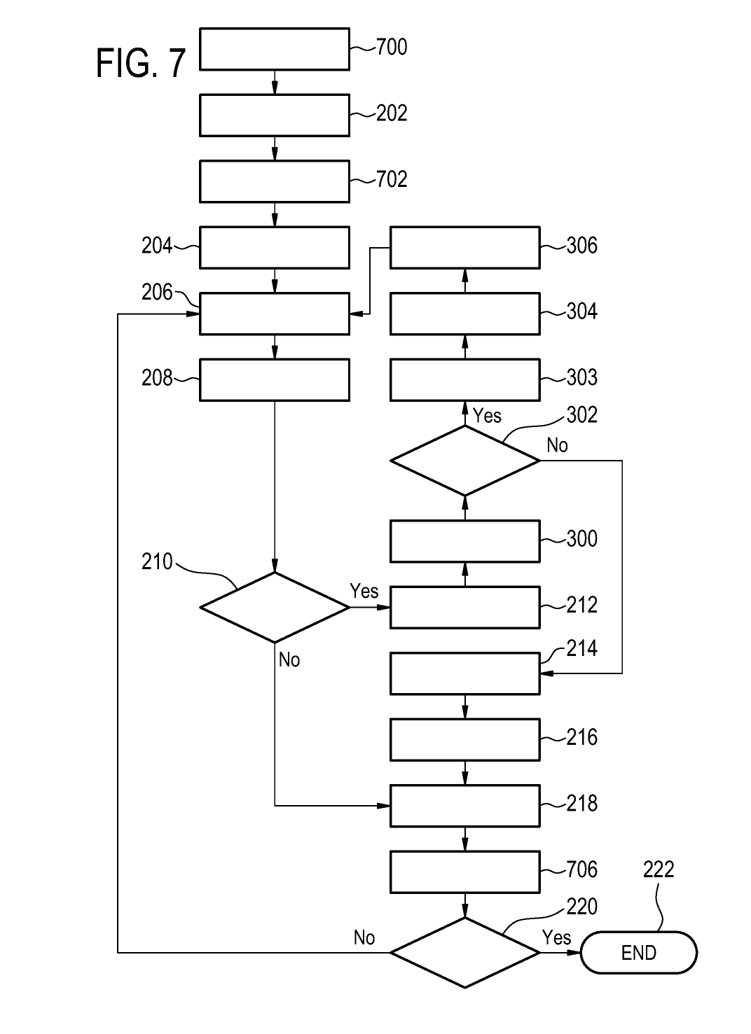

FIG. 7 shows a further example of a flowchart which illustrates a method of operating the medical instruments of FIG. 4, 5 or 6. The method shown in FIG. 7 is similar to the method of FIG. 3 with several modifications. The differences are that a step 700 is performed before step 202 and in this example method the method proceeds from step 202 to step 702 and then to step 204. The method also proceeds from step 218 to step 706 and then to step 220. First in step 700 temperature control system commands are received. These for example could be received via a network connection or could be entered manually for example by a physician or other operator. The method then proceeds to step 202 of FIG. 3. After step 202 is performed the method then proceeds to step 702. In step 702 the processor controls the temperature control system to the region 420. The method could also be performed without step 702 being performed. In this case the method would proceed directly from step 202 to step 204. After step 702 is performed the method proceeds to the step 204 of FIG. 3. In step 218 the PRFS temperature map is calculated. Next the method proceeds to step 706. In step 706 the temperature control system commands are modified using the PRFS temperature map 162. After the commands have been modified the method then normally proceeds to step 220 of FIG. 3.

PRFS temperature mapping is state-of-the-art during clinical MR-HIFU ablation, but for a long sonication time as in hyperthermia PRFS temperature maps are subject to errors due to B0 drift. A new acquisition and reconstruction for independent concurrent T1-based temperature mapping is proposed to correct for such drift. It is based on an interleaved T1 and PRFS sequence. The T1 sequence may be a saturation recovery Look-locker-type sequence to reset the spin history from previous PRFS. It is proposed to acquire the lacking M0 information for T1 reconstruction in a separate scan immediately before the start of the dynamic interleaved sequence (M0 scan) when the magnetization is still in equilibrium. It is proposed how to correct for motion which may occur between the M0 scan and the dynamic acquisitions, because such motion would introduce errors in the pixel-wise calculation of T1. Each latest image acquired during the T1 relaxation after saturation is compared to the M0 scan. The difference is evaluated by a cross-correlation (deriving a rigid body motion), or by an elastic registration, or by an optical flow algorithm. If the motion since the M0 scan exceeds a certain threshold, the dynamic sequence for example may be stopped for about 5*T1 to allow equilibrium magnetization to build up. Then, the M0 scan is repeated and dynamic interleaved imaging commences. If clinically required, HIFU sonication is stopped in that unsupervised period. The T1 map delivers independent temperature information, which is used to correct for the B0 drift.

MR guided high-intensity focused ultrasound (MR-HIFU) is establishing as a new treatment option for various diseases that elegantly combines two non-invasive technologies. Treatment options include HIFU ablation as well as adjuvant HIFU hyperthermia—precisely controlled by MR temperature mapping to adjust the applied HIFU acoustic power and focal spot position in real-time. Currently, temperature mapping based on the proton resonance frequency shift (PRFS) is applied during clinical MR-HIFU treatment. HIFU hyperthermia requires long sonication times (>20 min) and concurrent temperature mapping. PRFS-based temperature maps are subject to errors that increase over time because an unknown B0 drift B0(r) renders the reference phase map outdated after some time. The reacquisition of the reference map is not possible since the tissue is already heated. Hence, it is advantageous to derive the drift by measuring the temperature independently, e.g. by a T1 map, and to exploit the known temperature dependence of T1.

Modern scan software allows fast interleaving of different imaging sequences with microsecond latency. For above HIFU application, a dynamic T1 sequence should be interleaved with the PRFS sequence. The original versions of fast T1 mapping sequences follow the T1 relaxation after an inversion with small flip angle excitations (Look-Locker) to minimally disturb relaxation. A correction for this disturbance is known and can be applied.

The above Inversion Recovery (IR)-based T1 mapping sequences can in principle be combined with k-space segmentation and interleaved with PRFS acquisitions to derive a dynamic sequence. However, the IR-based approach does not work in such interleaved dynamic sequences since the IR scheme requires equilibrium magnetization M0 to be present at the time of inversion. Any previous PRFS acquisition disturbs this state. A known solution to this problem is to use a saturation-recovery-based variant which makes the subsequent T1 relaxation independent from the spin history (e.g. from preceding PRFS scans) in interleaved scanning. However, such sequences in principle cannot derive any information on M0, which is required to reconstruct for T1. This was solved by adding a saturation preparation as additional pre-pulse followed by a waiting time and then the original IR-prepared TFE-EPI sequence. This “clears” the spin history and leaves M0 information, but the waiting time of about 2*T1 (longest abdominal T1 is 1.5 s) effectively almost doubles the overall acquisition time. This concept is hence not applicable to dynamic interleaved scanning for HIFU.

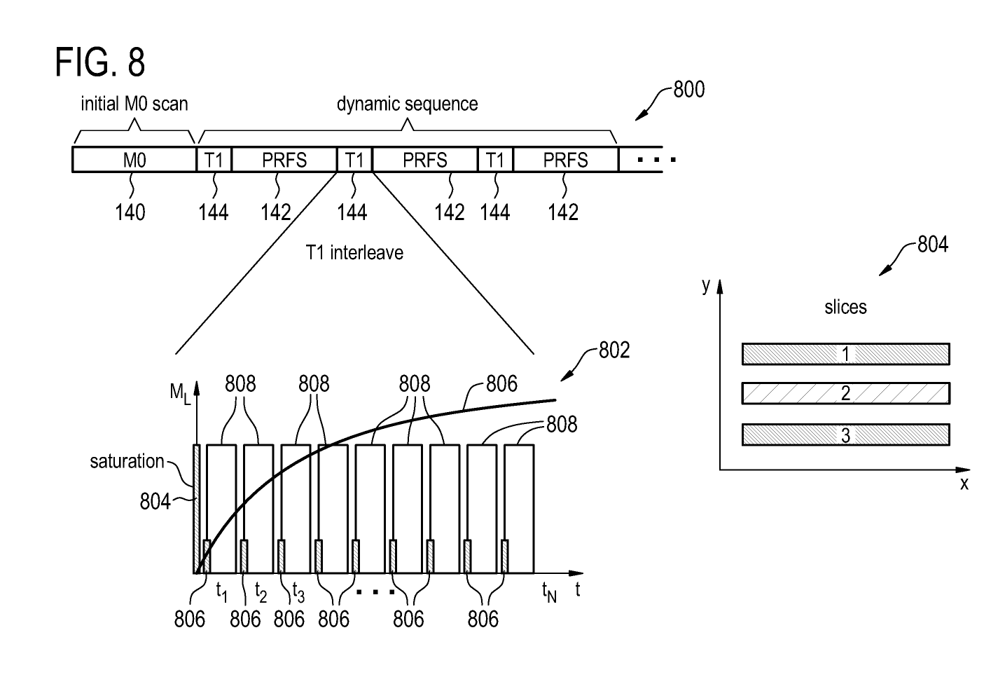

A new acquisition scheme and reconstruction is proposed that avoids additional acquisition time during interleaved scanning. The T1 sequence is based on a pure saturation recovery followed by a Look-locker-type sequence. It is dynamically interleaved with a standard PRFS sequence which acquires the same slice and potentially additional slices (c.f. FIG. 8 below). The T1 sequence is segmented such that after one saturation preparation, at each time point ti after the saturation one of M k-space segments is acquired. M T1 interleaves together fill N k-spaces acquired at the different times tᵢ.

It is possible to acquire the lacking M0 information in a separate scan immediately before the start of the dynamic interleaved sequence (M0 scan). At this time, the magnetization is still in equilibrium. The M0 scan is proposed to be mostly identical to one T1 interleave, however without saturation preparation, and with a different k-space acquisition order to acquire a full image in one interleave. A low-high k-space order is used with the central k-space segment acquired in the first EPI train (c.f. FIG. 2). This ensures that image contrast is dominated from the equilibrium magnetization, while the excitation pulses used for the subsequent EPI trains/k-space segments are already slightly disturbing equilibrium. Flip angle, TR, TE, EPI-factor and all other sequence parameters of the M0 scan should be chosen as in the acquisition in the subsequent T1 interleaves of the dynamic sequence.

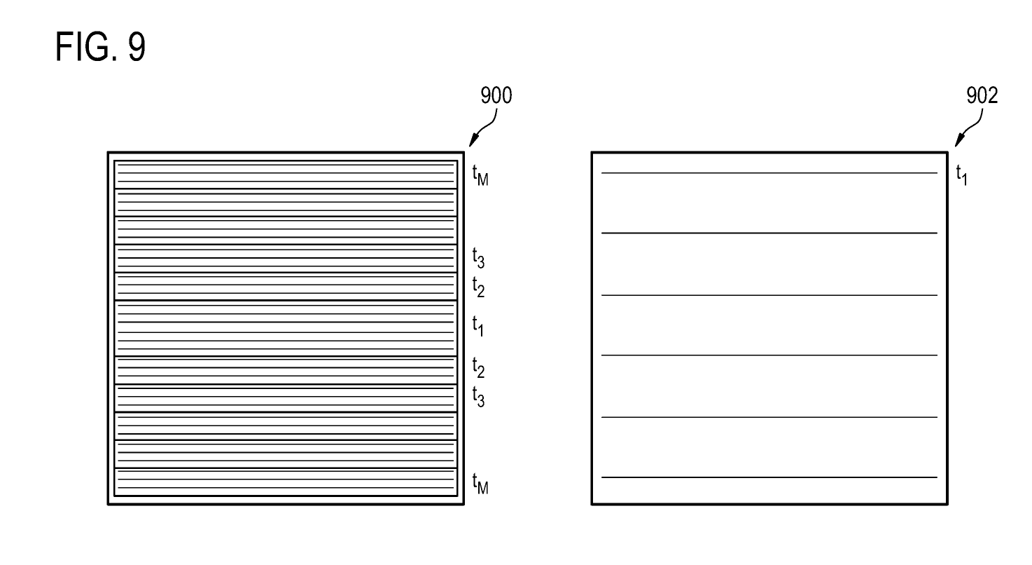

FIG. 8 illustrates the combined pulse sequence 800. The combined pulse sequence illustrates how the first pulse sequence commands 140 are performed initially and then the second pulse sequence commands 142 and the third pulse sequence commands 144 are performed in an interleaved manner. During each of the executions of the third pulse sequence commands 144 the k-space trajectory for each time is separate such that eventually the entire set of magnetic resonance data portions is acquired and the dynamic T1 magnetic resonance data can be reconstructed. The plot 802 illustrates the acquisition of dynamic T1 magnetic resonance data. This is a timing diagram that shows that initially a radio-frequency saturation preparation 804 is performed. This brings the longitudinal magnetization 806 to 0. As time progresses it can be seen that the magnetization 806 recovers. During each position of the dynamic T1 magnetic resonance data the plots 806 illustrate the first radio-frequency pulse used to indicate when data is acquired and the boxes 808 illustrate a time window when the data are acquired according to FIG. 9 (see image 902). FIG. 9 shows the k-space order of equilibrium magnetization magnetic resonance data 900 and also the k-space order 902 of the magnetic resonance data portion of the dynamic T1 magnetic resonance data. This illustrates the difference in the acquisition of data between the first pulse sequence 140 and the third pulse sequence commands 144. During each of the executions 144 only a portion of the k-space is acquired since the data should be confined to a specific time window 808 along the relaxation curve. However, it should be noted that the k-space data is acquired along a particular trajectory which enables both the central and outer regions of k-space to be sampled. After collection of several such portions, a new image representing the object at a late time along the relaxation curve can be calculated and used for motion detection. This provides a means of detecting motion in addition to calibrating the PRFS temperature measurements.

A method of performing the T1 reconstruction:

Firstly, the M0 scan may be reconstructed by a standard reconstruction and provides an undisturbed image with a signal proportional to M0 (apart from factors that are identical to the later acquisition as T2 relaxation terms).

During the dynamic phase, complete sets of M dynamic T1 interleaves (characterized by the fact that the entire k-space is covered for all N time points sampled on the relaxation curve) are used to reconstruct a series of N images with effective acquisition times ti (i=1 . . . N) during relaxation after the saturation preparation.

A pixel-wise three parameter fit [ ] is used to estimate the parameters M(0), M0*, and T1* according to:

$$M ( {t}_{i} ) = {M0}^{*} - ( {M0}^{*} - M ( 0 ) ) {e}^{\frac{{t}_{i}}{{T}_{1}^{*}}}$$

The apparent T1* is shorter than T1, and T1 can be calculated by

$${T}_{1} = - {τ [ ln ( 1 - \frac{{M0}^{*}}{M 0} ( 1 - {e}^{- \frac{τ}{{T}_{1}^{*}}} ) ) ]}^{- 1}$$

for each pixel, where it is proposed to use the respective pixel value in the image of the M0 scan for M0. M(0) is treated as fit parameter here to account for imperfections of the saturation preparation that may lead to a non-zero initial magnetization. M(0) may be assumed to be zero otherwise, leading to a two parameter fit.

The strategy to acquire M0 information only once before the dynamic acquisition raises the problem to cope with motion which may occur between the M0 acquisition and the dynamic acquisitions. Such motion will introduce errors in the pixel-wise calculation of T1.

It is therefore proposed to check during the dynamic acquisition whether the M0 image has become outdated by motion as follows: Each dynamic image M(N) reconstructed from the last time point of the relaxation is expected to be very similar in contrast to the M0 scan. The images M(N) and M0 are evaluated to derive a displacement field that describes the in-plane motion that has occurred since the M0 scan. This evaluation is proposed to be a simple cross-correlation (deriving a rigid body motion), an elastic registration, or an optical flow algorithm. Alternatively, a state-of-the-art similarity measure may be used to derive the similarity between M(N) and M0. If the motion since the M0 scan exceeds or the similarity between M(N) and M0 falls below a user-defined threshold, the M0 scan is outdated. Hence, the dynamic sequence must be stopped for about 5*T1, i.e. about 5 s to allow equilibrium magnetization to build up. Then, the M0 scan is repeated and dynamic imaging commences. If clinically required, HIFU sonication must be stopped in that unsupervised period.

It is also possible to increase the temporal resolution of the dynamic series of T1 images by using a sliding window approach: For every acquired T1 interleave (providing a new segment of k-space lines for each time point ti), a new T1 map is reconstructed with the newest set of M interleaves, effectively replacing the respective outdated set of k-space lines.

Example parameters for some pulse sequences:

T1 sequence:

non-sel SR-prep T1w-TFE; TFE shots M=5; TFE factor=20; SENSE-P=1.8; FOV=250×250 mm2; resolution=1.42×1.42 mm2; slice thickness=4 mm; Tacq per interleave=2000 ms with N=12 time points along relaxation)

PRFS sequence:

M2D T1w-FFE-EPI, TR/TE=41/19.5 ms; flip angle=19.5°; EPI factor=7; SENSE-P=1.8; FOV=250×250 mm2; resolution=1.42×1.42 mm2; 3 slices; NSA=2; fat suppression; dynamic acq. time=5.4 s

Correction of PRFS temperature map:

With the known temperature dependence of T1, an independent temperature map of slice 2 (c.f. FIG. 1) is calculated after every M T1 interleaves (or even after each interleave in case of the sliding window reconstruction). This temperature map is compared with the map derived from the dynamic PRFS sequence which is reconstructed as known in the art. Differences that will occur due to the B0 drift B0(r) between both temperature maps are used to correct the PRFS temperature maps by setting a respective reference phase for the PRFS reconstruction as known in the art.

While the invention has been illustrated and described in detail in the drawings and foregoing description, such illustration and description are to be considered illustrative or exemplary and not restrictive; the invention is not limited to the disclosed embodiments.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measured cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

### LIST OF REFERENCE NUMERALS

100 medical instrument
102 magnetic resonance imaging system
104 magnet
106 bore of magnet
108 imaging zone
110 magnetic field gradient coil
112 magnetic field gradient coil power supply
114 radio frequency coil
116 radio frequency transceiver
118 subject
120 subject support
122 computer system
124 hardware interface
126 processor
128 user interface
130 computer storage
132 computer memory
140 first pulse sequence commands
142 second pulse sequence commands
144 third pulse sequence commands
148 equilibrium magnetization magnetic resonance data
150 dynamic PRFS magnetic resonance data
152 magnetic resonance data portion
154 reassembled dynamic T1 magnetic resonance data
156 equilibrium magnetization baseline image
158 T1 map
160 PRFS phase calibration
162 PRFS temperature map
170 Control module
172 Image reconstruction module
174 Image processing module
176 temperature mapping module
202 acquire equilibrium magnetization magnetic resonance imaging data by controlling the magnetic resonance imaging system using the first pulse sequence commands
204 calculate an equilibrium magnetization baseline image from the equilibrium magnetization magnetic resonance imaging data
206 acquire the PRFS magnetic resonance data by controlling the magnetic resonance imaging system with the second pulse sequence commands;
208 acquire a magnetic resonance data portion by controlling the magnetic resonance imaging system with the third pulse sequence commands
210 all data portions of the dynamic T1 magnetic resonance data acquired?
212 reassemble the set of magnetic resonance data portions into the dynamic T1 magnetic resonance data after the complete set of magnetic resonance data portions is acquired
214 calculate a T1 map using the reassembled dynamic T1 magnetic resonance data and the equilibrium magnetization image
216 calculate a PRFS phase calibration using the PRFS magnetic resonance data and the T1 map
218 calculate a PRFS temperature map using the PRFS magnetic resonance data and the PRFS phase calibration if the PRFS phase calibration has been calculated
220 protocol complete?
300 calculate a dynamic image from the magnetic resonance data portion
302 motion detected?
303 pause
304 reacquire equilibrium magnetization magnetic resonance imaging data by controlling the magnetic resonance imaging system using the first pulse sequence commands if the subject motion is detected
306 recalculate the equilibrium magnetization baseline image from the equilibrium magnetization magnetic resonance imaging data if the subject motion is detected.
400 medical apparatus
402 high intensity focused ultrasound system
404 fluid filled chamber
406 ultrasound transducer
408 mechanism
410 mechanical actuator/power supply
412 path of ultrasound
414 ultrasound window
416 gel pad
418 sonication point
420 target zone
430 dynamic image
432 temperature control system commands
440 motion detection module
442 temperature control system command modification module
500 medical apparatus
501 radio-frequency tissue heating system
502 antenna
504 radio-frequency transmitter
600 medical apparatus
601 thermal treatment system
602 applicator
604 supply system
700 receive temperature control system commands that cause the temperature control system to modify the temperature of the target zone
702 control the temperature control system with the temperature control system commands 706 modify the temperature control system commands using the PRFS temperature map
800 combined pulse sequence
802 illustration of acquisition of dynamic T1 data
804 saturation preparation
806 acquisition of data
808 spatial encoding
900 k-space order of equilibrium magnetization magnetic resonance data
902 k-space order of magnetic resonane data portion of the dynamic T1 magnetic resonance data

1. A medical instrument comprising,
a magnetic resonance imaging system for acquiring magnetic resonance data within an imaging zone;
a memory storing machine executable instructions, first pulse sequence commands, second pulse sequence commands, and third pulse sequence commands, wherein the first pulse sequence commands cause the magnetic resonance imaging system to acquire equilibrium magnetization magnetic resonance data according to a T1 measuring magnetic resonance imaging protocol, wherein the second pulse sequence commands cause the magnetic resonance imaging system to acquire dynamic PRFS magnetic resonance data according to a proton resonance frequency shift magnetic resonance imaging protocol, wherein the third pulse sequence commands cause the magnetic resonance imaging system to acquire dynamic T1 magnetic resonance data according to the T1 measuring magnetic resonance imaging protocol, wherein the third pulse sequence commands further cause the magnetic resonance imaging system to acquire the dynamic T1 magnetic resonance data sequentially as a set of magnetic resonance data portions, wherein the third pulse sequence commands cause the magnetic resonance imaging system to perform a saturation preparation at the start of the acquisition of each magnetic resonance data portion,
a processor for controlling the medical instrument, wherein execution of the machine executable instructions causes the processor to:
acquire equilibrium magnetization magnetic resonance imaging data by controlling the magnetic resonance imaging system using the first pulse sequence commands;
calculate an equilibrium magnetization baseline image from the equilibrium magnetization magnetic resonance imaging data;
wherein execution of the machine executable instructions causes the processor to repeatedly:
acquire the dynamic PRFS magnetic resonance data by controlling the magnetic resonance imaging system with the second pulse sequence commands;
acquire a magnetic resonance data portion by controlling the magnetic resonance imaging system with the third pulse sequence commands, wherein the acquisition of the dynamic PRFS magnetic resonance data and the magnetic resonance data portion are interleaved, wherein the magnetic resonance data portion belongs to the set of magnetic resonance data portions;
reassemble the set of magnetic resonance data portions into the dynamic T1 magnetic resonance data after the complete set of magnetic resonance data portions is acquired;
calculate a T1 map using the reassembled dynamic T1 magnetic resonance data and the equilibrium magnetization image, wherein the equilibrium magnetization image is reconstructed from data that was acquired before the dynamic PRFS magnetic resonance data was acquired;
calculate a PRFS phase calibration using the dynamic PRFS magnetic resonance data and the T1 map; and
calculate a PRFS temperature map using the dynamic PRFS magnetic resonance data and the PRFS phase calibration if the PRFS phase calibration has been calculated.

2. The medical instrument of claim 1, wherein execution of the instructions further causes the processor to repeatedly:
calculate a dynamic image from the magnetic resonance data portion;
detect a subject motion above a predetermined threshold using the equilibrium magnetization baseline image and the dynamic image,
reacquire the equilibrium magnetization magnetic resonance imaging data by controlling the magnetic resonance imaging system using the first pulse sequence commands if the subject motion is detected,
recalculate the equilibrium magnetization baseline image from the equilibrium magnetization magnetic resonance imaging data if the subject motion is detected.

3. The medical instrument of claim 2, wherein the subject motion is detected using any one of the following: a cross correlation algorithm, a rigid body motion detection algorithm, an elastic registration algorithm, optical flow algorithm, and combinations thereof.

4. The medical instrument of claim 1, wherein the medical instrument further comprises a temperature control system for modifying the temperature within a target zone, wherein the target zone is within the imaging zone.

5. The medical instrument of claim 4, wherein the temperature control system is any one of the following: a high intensity focused ultrasound system, a radio-frequency tissue heating system, microwave applicator, a cryo-ablator, and a laser.

6. The medical instrument of claim 4, wherein execution of the machine executable instructions further cause the processor to:
receive temperature control system commands that cause the temperature control system to modify the temperature of the target zone, and
repeatedly modify the temperature control system commands using the PRFS temperature map.

7. The medical instrument of claim 6, wherein the medical instrument further comprises a user interface with a display, wherein execution of the machine executable instructions further cause the processor to:
display the PRFS temperature map on the display;
receive user control data from the user interface; and
modify the temperature control system commands using the user control data.

8. The medical instrument of claim 6, wherein execution of the machine executable instructions causes the processor to control the temperature control system with the temperature control system commands.

9. The medical instrument of claim 1, wherein the T1 measuring magnetic resonance imaging protocol is a saturation recovery Look-locker magnetic resonance imaging protocol.

10. The medical instrument of claim 1, wherein execution of the magnetic resonance imaging system causes the processor to perform the following a predetermined time interval after acquiring the equilibrium magnetization magnetic resonance imaging data:
reacquire the equilibrium magnetization magnetic resonance imaging data by controlling the magnetic resonance imaging system using the first pulse sequence commands, and
recalculate an equilibrium magnetization image from the equilibrium magnetization magnetic resonance imaging data if the subject motion is detected.

11. A method of operating a medical instrument, wherein the medical instrument comprises a magnetic resonance imaging system for acquiring magnetic resonance data within an imaging zone; wherein the method comprises the steps of:
acquiring equilibrium magnetization magnetic resonance imaging data by controlling the magnetic resonance imaging system using first pulse sequence commands, wherein the first pulse sequence commands cause the magnetic resonance imaging system to acquire equilibrium magnetization magnetic resonance data according to a T1 measuring magnetic resonance imaging protocol;
calculating an equilibrium magnetization baseline image from the equilibrium magnetization magnetic resonance imaging data;
the method further comprises repeatedly:
acquiring the dynamic PRFS magnetic resonance data by controlling the magnetic resonance imaging system with second pulse sequence commands, wherein the second pulse sequence commands cause the magnetic resonance imaging system to acquire the dynamic PRFS magnetic resonance data according to a proton resonance frequency shift magnetic resonance imaging protocol;
acquiring a magnetic resonance data portion by controlling the magnetic resonance imaging system with third pulse sequence commands, wherein the third pulse sequence commands cause the magnetic resonance imaging system to acquire dynamic T1 magnetic resonance data according to the T1 measuring magnetic resonance imaging protocol, wherein the third pulse sequence commands further cause the magnetic resonance imaging system to acquire the dynamic T1 magnetic resonance data sequentially as a set of magnetic resonance data portions, wherein the acquisition of the dynamic PRFS magnetic resonance data and the magnetic resonance data portion are interleaved, wherein the magnetic resonance data portion belongs to the set of magnetic resonance data portions, wherein the third pulse sequence commands cause the magnetic resonance imaging system to perform a saturation preparation at the start of the acquisition of each magnetic resonance data portion;
reassembling the set of magnetic resonance data portions into the dynamic T1 magnetic resonance data after the complete set of magnetic resonance data portions is acquired;
calculating a T1 map using the reassembled dynamic T1 magnetic resonance data and the equilibrium magnetization image, wherein the equilibrium magnetization image is reconstructed from data that was acquired before the dynamic PRFS magnetic resonance data was acquired;
calculating a PRFS phase calibration using the dynamic PRFS magnetic resonance data and the T1 map; and
calculating a PRFS temperature map using the dynamic PRFS magnetic resonance data and the PRFS phase calibration if the PRFS phase calibration has been calculated.

12. The method of claim 11, wherein the method further comprises repeatedly:
calculating a dynamic image from a chosen magnetic resonance data selected from the set of magnetic resonance data portion, wherein the chosen magnetic resonance data portion is chosen to maximize the longitudinal magnetization;
detecting a subject motion above a predetermined threshold using the equilibrium magnetization baseline image and the dynamic image,
reacquiring the equilibrium magnetization magnetic resonance imaging data by controlling the magnetic resonance imaging system using the first pulse sequence commands if the subject motion is detected,
recalculating the equilibrium magnetization baseline image from the equilibrium magnetization magnetic resonance imaging data if the subject motion is detected.

13. The method of claim 12, wherein the subject motion is detected using any one of the following: a cross correlation algorithm, a rigid body motion detection algorithm, an elastic registration algorithm, optical flow algorithm, and combinations thereof.

14. A computer program product comprising machine executable instructions for execution by a processor controlling a medical instrument, wherein the medical instrument comprises a magnetic resonance imaging system for acquiring magnetic resonance data within an imaging zone, wherein execution of the machine executable instructions causes the processor to:
acquire equilibrium magnetization magnetic resonance imaging data by controlling the magnetic resonance imaging system using first pulse sequence commands, wherein the first pulse sequence commands cause the magnetic resonance imaging system to acquire the equilibrium magnetization magnetic resonance data according to a T1 measuring magnetic resonance imaging protocol;
calculate an equilibrium magnetization baseline image from the equilibrium magnetization magnetic resonance imaging data;
wherein execution of the machine executable instructions causes the processor to repeatedly:
acquire dynamic PRFS magnetic resonance data 150 by controlling the magnetic resonance imaging system with the second pulse sequence commands, wherein the second pulse sequence commands cause the magnetic resonance imaging system to acquire the dynamic PRFS magnetic resonance data according to a proton resonance frequency shift magnetic resonance imaging protocol;
acquire a magnetic resonance data portion by controlling the magnetic resonance imaging system with third pulse sequence commands, wherein the third pulse sequence commands cause the magnetic resonance imaging system to acquire dynamic T1 magnetic resonance data according to the T1 measuring magnetic resonance imaging protocol, wherein the third pulse sequence commands further cause the magnetic resonance imaging system to acquire the dynamic T1 magnetic resonance data sequentially as a set of magnetic resonance data portions, wherein the acquisition of the dynamic PRFS magnetic resonance data and the magnetic resonance data portion are interleaved, wherein the magnetic resonance data portion belongs to the set of magnetic resonance data portions, wherein the third pulse sequence commands cause the magnetic resonance imaging system to perform a saturation preparation at the start of the acquisition of each magnetic resonance data portion;
reassemble the set of magnetic resonance data portions into the dynamic T1 magnetic resonance data after the complete set of magnetic resonance data portions is acquired;
calculate a T1 map using the reassembled dynamic T1 magnetic resonance data and the equilibrium magnetization image, wherein the equilibrium magnetization image is reconstructed from data that was acquired before the dynamic PRFS magnetic resonance data was acquired;
calculate a PRFS phase calibration using the dynamic PRFS magnetic resonance data and the T1 map; and
calculate a PRFS temperature map using the dynamic PRFS magnetic resonance data and the PRFS phase calibration if the PRFS phase calibration has been calculated.
